(19) United States
(12) Patent Application Publication
(10) Pub. No.: US 2026/0279567 A1
(43) Pub. Date: Sep. 17, 2026

(54) SKIN DIAGNOSTIC SYSTEM, SKIN DIAGNOSTIC APPARATUS, NON-TRANSITORY COMPUTER-READBLE RECORDING MEDIUM STORING PROGRAM, AND METHOD

(71) Applicant: SHISEIDO COMPANY, LTD., Tokyo (JP)

(72) Inventors: Go TAKEHI, Tokyo (JP); Tetsuro YONEZAWA, Tokyo (JP); Naomi KANNO, Tokyo (JP); Yuki TAKAHASHI, Tokyo (JP); Yuusuke HARA, Tokyo (JP); Motoki TAKATA, Tokyo (JP)

(21) Appl. No.: 19/135,222

(22) PCT Filed: Dec. 4, 2023
(86) PCT No.: PCT/JP2023/043350

§ 371 Date: Jun. 5, 2026

(30) Foreign Application Priority Data
Dec. 20, 2022 (JP) 2022-203479

(51) Int. Cl. G16H 50/20; G16H 20/00; G16H 30/40; G16H 40/67

(52) U.S. Cl. CPC G16H 50/20; G16H 20/00; G16H 30/40; G16H 40/67

(57) ABSTRACT

To improve skin diagnoses, a skin diagnostic system according to an embodiment of the present invention is a skin diagnostic system including a terminal and a server, and includes: a skin condition acquisition unit configured to acquire a skin condition of a user; a blood vessel condition acquisition unit configured to acquire a blood vessel condition of the user; and a skin index calculation unit configured to calculate a skin index of the user based on the skin condition acquired and the blood vessel condition acquired.

### TECHNICAL FIELD

The present invention relates to a skin diagnostic system, a skin diagnostic apparatus, a program, and a method.

### BACKGROUND ART

Conventionally, it has been known to diagnose the surface condition of skin. For example, the surface condition of skin is diagnosed using a photographed image of the skin.

### CITATION LIST

### Patent Literature

PTL 1: Japanese Patent Application Laid-Open Publication No. 2014-219781

### SUMMARY OF THE INVENTION

### Technical Problem

However, it has been desired that not only the surface condition of a user's skin but also the internal condition of the user's skin, which affects the future condition of the skin, can be diagnosed.

Therefore, it is an object of the present invention to improve skin diagnoses.

### Solution to the Problem

A skin diagnostic system according to an embodiment of the present invention is a skin diagnostic system, including a terminal and a server, and includes: a skin condition acquisition unit configured to acquire a skin condition of a user; a blood vessel condition acquisition unit configured to acquire a blood vessel condition of the user; and a skin index calculation unit configured to calculate a skin index of the user based on the skin condition acquired and the blood vessel condition acquired.

### Advantageous Effects of the Invention

According to the present invention, skin diagnoses can be improved.

### BRIEF DESCRIPTION OF THE DRAWINGS

FIG. 1 is an example of the overall configuration of an embodiment of the present invention.

FIG. 2 is a functional block diagram of a skin diagnostic apparatus according to an embodiment of the present invention.

FIG. 3 is an example of information stored in a recommendation information storage unit according to an embodiment of the present invention.

FIG. 4 is a display example of calculated results of skin indices according to an embodiment of the present invention.

FIG. 5 is a display example of calculated results of skin indices according to an embodiment of the present invention.

FIG. 6 is a display example of calculated results of skin indices according to an embodiment of the present invention.

FIG. 7 is a display example of a blood vessel condition according to an embodiment of the present invention.

FIG. 8 FI. 8 is a display example of a blood vessel condition according to an embodiment of the present invention.

FIG. 9 is a flowchart of a skin diagnostic process according to an embodiment of the present invention.

FIG. 10 is a hardware configuration diagram of a skin diagnostic apparatus and a terminal according to an embodiment of the present invention.

### DETAILED DESCRIPTION OF THE INVENTION

Hereinafter, an embodiment of the present invention will be described based on the drawings.

### Explanation of Terms

As used herein, the term “skin condition” refers to a surface condition of the skin of a human face (i.e., the current appearance of the skin). For example, the skin condition includes “blood flow circulation”, “wrinkle and flabbiness”, and “skin unevenness”.
As used herein, the term “skin index” refers to an index of a skin factor (skin element) of a human face. According to the present invention, the skin index can be calculated based not only on the skin conditions (i.e., the current appearance of the skin) but also on the internal condition of the skin (specifically, the blood vessel condition), which affects the future skin. For example, the “skin index” includes “transparency”, “firmness/resilience”, and “smoothness”.

### Example of Overall Configuration

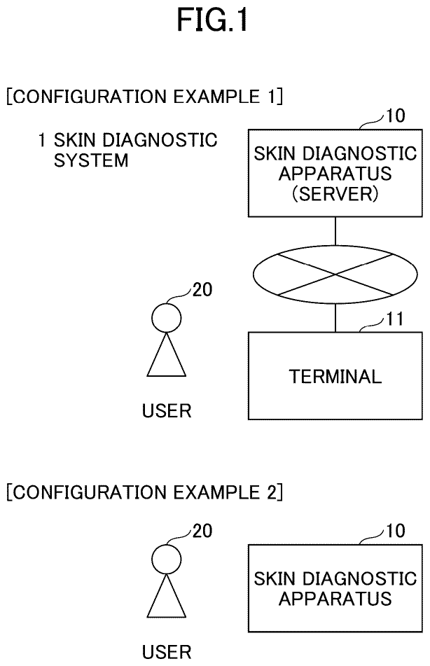

FIG. 1 is an example of the overall configuration according to an embodiment of the present invention.

### [Configuration Example 1]

A skin diagnostic system 1 includes a skin diagnostic apparatus (e.g., a server) 10 and a terminal 11. The skin diagnostic apparatus 10 and the terminal 11 are communicably connected through a desired network. In Configuration Example 1, a user 20 (or a beauty consultant of a cosmetics shop or the like) operates the terminal 11. Each will be described below.

### <<Skin Diagnostic Apparatus>>

The skin diagnostic apparatus 10 is an apparatus for calculating the skin index of the user 20 based on the skin condition and the blood vessel condition of the user 20. For example, the skin diagnostic apparatus 10 is a server or the like composed of one or a plurality of computers.

### <<Terminal>>

The terminal 11 acquires the skin condition and the blood vessel condition of the user 20, transmits them to the skin diagnostic apparatus 10, and receives and displays the diagnostic results of the skin diagnostic apparatus 10. The terminal 11 may perform a part of the processes of the skin diagnostic apparatus 10 described herein.

For example, the terminal 11 transmits to the skin diagnostic apparatus 10, an image of the facial skin of the user 20 captured by a photographing camera function, data of the temperature of the facial skin of the user 20 acquired by the thermographic camera (also referred to as thermo-camera or thermistor camera) function, and an answer by the user 20 to an inquiry (a questionnaire about the skin condition).

For example, the terminal 11 may be a smart mirror (specifically, a display having a photographing camera function and a thermographic camera function) installed in a store, a smartphone having a photographing camera function and a thermographic camera function, a tablet having a photographing camera function and a thermographic camera function, and the like. A terminal 11 that does not have a photographing camera function and a thermographic camera function may acquire a facial skin image and facial skin temperature data photographed and acquired by a device having a photographing camera function and a thermographic camera function.

### [Configuration Example 2]

In Configuration Example 2, the user 20 (or a beauty consultant of a cosmetics shop or the like) operates the skin diagnostic apparatus 10.

### <<Skin Diagnostic Apparatus>>

The skin diagnostic apparatus 10 is an apparatus for calculating the skin index of the user 20 based on the skin condition and the blood vessel condition of the user 20. For example, the skin diagnostic apparatus 10 may be a smart mirror (specifically, a display having a photographing camera function and a thermographic camera function) installed in a store, a smartphone having a photographing camera function and a thermographic camera function, a tablet having a photographing camera function and a thermographic camera function, and the like. A skin diagnostic apparatus 10 that does not have a photographing camera function and a thermographic camera function may acquire a facial skin image and facial skin temperature data photographed and acquired by a device having a photographing camera function and a thermographic camera function.

### <Function Block>

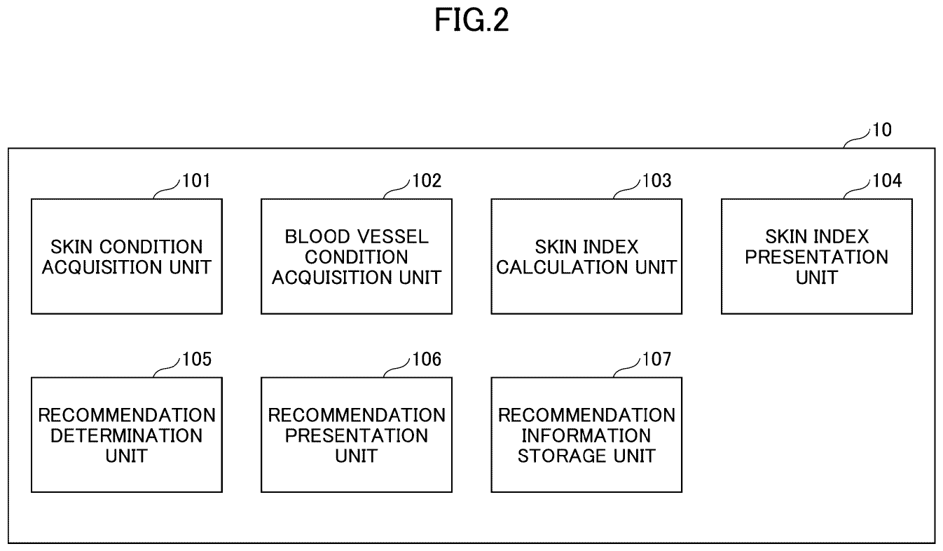

FIG. 2 is a functional block diagram of the skin diagnostic apparatus 10 according to an embodiment of the present invention. The skin diagnostic apparatus 10 includes a skin condition acquisition unit 101, a blood vessel condition acquisition unit 102, a skin index calculation unit 103, a skin index presentation unit 104, a recommendation determination unit 105, a recommendation presentation unit 106, and a recommendation information storage unit 107. The skin diagnostic apparatus 10 functions as the skin condition acquisition unit 101, the blood vessel condition acquisition unit 102, the skin index calculation unit 103, the skin index presentation unit 104, the recommendation determination unit 105, and the recommendation presentation unit 106 by executing a program.

The skin condition acquisition unit 101 acquires the skin condition (e.g., the surface condition of the skin) of the user 20. For example, the skin condition acquisition unit 101 can evaluate the skin condition of the user 20 based on at least one of a photographed image of the facial skin of the user 20, the temperature of the facial skin of the user 20, or an answer by the user 20 to an inquiry (a questionnaire about the skin condition), and utilize the skin condition.

### [Skin Condition]

The skin condition will now be described. For example, the skin condition is at least one of “blood flow circulation” evaluated based on the skin temperature and the answer to the inquiry, “wrinkle and flabbiness” evaluated by a skin image analysis, or “skin unevenness” evaluated by a skin image analysis. For example, the skin condition may be a quantified score.

The blood vessel condition acquisition unit 102 acquires the blood vessel condition of the user 20. For example, the blood vessel condition acquisition unit 102 acquires skin information (e.g., number of blemishes, transepidermal water loss (TEWL), redness, yellowness, skin depression area ratio, texture nonuniformity, pore area ratio, collagen density, and the like) from a photographed image of the facial skin of the user 20, inputs the skin information into a model (e.g., a trained model trained by machine learning based on an error back propagation method or the like such that the blood vessel condition is output in response to an input of the skin information) to output the blood vessel condition. In this way, the blood vessel condition can be acquired.

### [Blood Vessel Condition]

The blood vessel condition will now be described. For example, the blood vessel condition is at least one of blood flow in capillary vessels, the number of capillary vessels, the density of capillary vessels, the number of capillary vessel branches, the distance from the base layer of the epidermis to the capillary vessels, or the thickness of the epidermis. For example, the blood vessel condition may be a quantified score. In the present invention, by using the internal condition of the skin (specifically, the blood vessel condition), which also affects the future condition of the skin, it is possible to diagnose the skin in consideration of not only the surface condition of the skin (i.e., the current appearance of the skin) but also the future condition of the skin.

The skin index calculation unit 103 calculates a skin index (e.g., quantified scores of “transparency”, “firmness/resilience”, and “smoothness”) of the user 20 based on the skin condition acquired by the skin condition acquisition unit 101 and the blood vessel condition acquired by the blood vessel condition acquisition unit 102.

For example, the skin index calculation unit 103 can switch the method for calculating the skin index of the user 20 between when the blood vessel condition of the user 20 is outside a predetermined range (e.g., when the blood vessel condition is not appropriate) and when the blood vessel condition of the user 20 is within the predetermined range (e.g., when the blood vessel condition is appropriate).

### [Predetermined Range of Blood Vessel Condition]

The predetermined range of the blood vessel condition will now be described. For example, the predetermined range of the blood vessel condition is a range in which the blood vessel condition is appropriate.

The skin index calculation unit 103 can set the predetermined range. For example, the skin index calculation unit 103 can set the predetermined range per attribute information, such as per age or age decade, per gender, per area, per lifestyle (e.g., spending a lot of time outdoors, spending a lot of time indoors, etc.), and the like.

For example, the skin index calculation unit 103 can set a value that is input by the user 20 or the like as the predetermined range. For example, the skin index calculation unit 103 can set a value that is input by the user 20 or the like per attribute information as the predetermined range. For example, the skin index calculation unit 103 can set a range including the median of the values of the blood vessel condition of a plurality of persons (i.e., a range that includes a number of persons that occupies a certain proportion from the median) as the predetermined range. For example, the skin index calculation unit 103 can set, per attribute information, a range including the median of the values of the blood vessel condition of a plurality of persons having the attribute information concerned as the predetermined range.

For example, the skin index calculation unit 103 can set a range including the median of the values of a related skin condition (i.e., a range that includes a number of persons that occupies a certain proportion from the median) as the predetermined range. For example, the skin index calculation unit 103 can set a range including the median of the values of the related skin condition as the predetermined range per attribute information.

Specifically, the blood vessel condition is correlated with the skin condition “blemish”, and “blemish” contributes to the transparency of the skin. The blood vessel condition is correlated with the skin condition “wrinkle”, and “wrinkle” contributes to the firmness and resilience of the skin. The blood vessel condition is correlated with the skin condition “transepidermal water loss (TEWL)”, and “transepidermal water loss (TEWL)” contributes to the smoothness of the skin. From the correlations between the blood vessel condition and the skin conditions “blemish”, “wrinkle”, and “transepidermal water loss (TEWL)”, the following formulae can be created.
Blood vessel condition=A×skin condition “blemish”+B
Blood vessel condition=C×skin condition “wrinkle”+D
Blood vessel condition=E×skin condition “transepidermal water loss (TEWL)”+F

The skin index calculation unit 103 can set, as the predetermined range, a range including the result returned when the median of the values of the skin condition “blemish”, “wrinkle”, or “transepidermal water loss (TEWL)” of a plurality of persons is input, the result being a blood vessel condition (of the case where the blemish median is input), a blood vessel condition (of the case where the wrinkle median is input), or a blood vessel condition (of the case where the “transepidermal water loss (TEWL)” median is input).

### [When Outside Predetermined Range]

When the blood vessel condition is outside the predetermined range, the skin index calculation unit 103 calculates the skin index of the user 20 based on the skin condition acquired by the skin condition acquisition unit 101 and the blood vessel condition acquired by the blood vessel condition acquisition unit 102. For example, the skin index calculation unit 103 calculates each index by adding or subtracting some points in accordance with the blood vessel condition score [specifically, by adding or subtracting the blood vessel condition score as follows: Skin index (transparency)=“blood flow circulation (skin condition evaluated based on skin moistness and an answer to an inquiry)”+α1×blood vessel condition score; Skin index (firmness/resilience)=“wrinkle/flabbiness (skin condition evaluated by a skin image analysis)”+α2×blood vessel condition score; Skin index (smoothness)=“skin unevenness (skin condition evaluated by a skin image analysis)”−α3×blood vessel condition score; and the like].

Note that α1, α2, and α3 may be different constants. For example, the values and positivity/negativity of α1, α2, and α3 can be determined in accordance with the degrees of the correlations between the skin factors that are the bases of the respective skin indices and the blood vessel conditions (e.g., the correlations between the skin factors, such as blemishes, wrinkle, transepidermal water loss (TEWL), and the like, and the blood vessel conditions, described below). For example, α1, α2, and α3 are large when the degrees of correlations with the blood vessel conditions are large, and α1, α2, and α3 are small when the degrees of correlations with the blood vessel conditions are small. Moreover, α1, α2, and α3 are positive when the degrees of correlations with the blood vessel conditions are positive, and α1, α2, and α3 are negative when the degrees of correlations with the blood vessel condition are negative.

For example, when a blood vessel condition score is high (e.g., when the blood flow velocity is high), the blemish score is high (i.e., there are less blemishes and the blemishes are pale), whereas when a blood vessel condition score is low (e.g., when the blood flow velocity is low), the blemish score is low (i.e., there are many blemishes, and the blemishes are dark). This indicates that the blood flow velocity contributes to the skin transparency.

For example, when a blood vessel condition score is high (e.g., when the blood flow velocity is high), the wrinkle score is high (i.e., there are less wrinkles, and the wrinkles are shallow), whereas when a blood vessel condition score is low (e.g., when the blood flow velocity is low), the wrinkle score is low (i.e., there are many wrinkles, and the wrinkles are deep). This indicates that the blood flow velocity contributes to the skin firmness and resilience.

For example, when a blood vessel condition score is high (e.g., when the blood flow velocity is high), the transepidermal water loss (TEWL) score is low (i.e., the skin is dry), whereas when a blood vessel condition score is low (e.g., when the blood flow velocity is low), the transepidermal water loss (TEWL) score is high (i.e., the skin is moist). This indicates that the blood flow velocity contributes to the skin smoothness.

### [When Within Predetermined Range]

When the blood vessel condition is within the predetermined range, the skin index calculation unit 103 calculates the skin index of the user 20 based on the skin condition acquired by the skin condition acquisition unit 101. For example, the skin index calculation unit 103 calculates the indices by adding some points to all skin indices [specifically, by adding β as follows: Skin index (transparency)=“blood flow circulation (skin condition evaluated based on skin moistness and an answer to an inquiry)”+β; Skin index (firmness/resilience)=“wrinkle/flabbiness (skin condition evaluated by a skin image analysis)”+β; Skin index (smoothness)=“skin unevenness (skin condition evaluated by a skin image analysis)”+β; and the like]. Here, β is a constant that may be selected as desired.

The skin index presentation unit 104 presents the skin index calculated by the skin index calculation unit 103 (e.g., by displaying on the skin diagnostic apparatus 10 or the terminal 11). For example, the skin index presentation unit 104 can present the contribution of the blood vessel condition to the skin index. For example, the skin index presentation unit 104 can present the contribution of the blood vessel condition to the skin index in a display mode corresponding to the skin condition acquired by the skin condition acquisition unit 101 and the blood vessel condition acquired by the blood vessel condition acquisition unit 102.

The recommendation determination unit 105 can determine a recommendation to be recommended to the user 20 based on both the skin condition acquired by the skin condition acquisition unit 101 and the blood vessel condition acquired by the blood vessel condition acquisition unit 102. Alternatively, the recommendation determination unit 105 can determine a recommendation to be recommended to the user 20 based only on the skin condition acquired by the skin condition acquisition unit 101, and a recommendation to be recommended to the user 20 based on both the skin condition acquired by the skin condition acquisition unit 101 and the blood vessel condition acquired by the blood vessel condition acquisition unit 102. The user 20 can select whether a recommendation should be determined based only on the skin condition or a recommendation should be determined based on both the skin condition and the blood vessel condition, and input an instruction into the skin diagnostic apparatus 10 or the terminal 11.

The recommendation presentation unit 106 presents the recommendation determined by the recommendation determination unit 105 to the user 20 (e.g., by displaying on the skin diagnostic apparatus 10 or the terminal 11).

The recommendation information storage unit 107 stores information regarding one or a plurality of recommendations corresponding to the skin condition and the blood vessel condition. The recommendation information storage unit 107 may store a recommendation based only on the skin condition and a recommendation based on both the skin condition and the blood vessel condition.

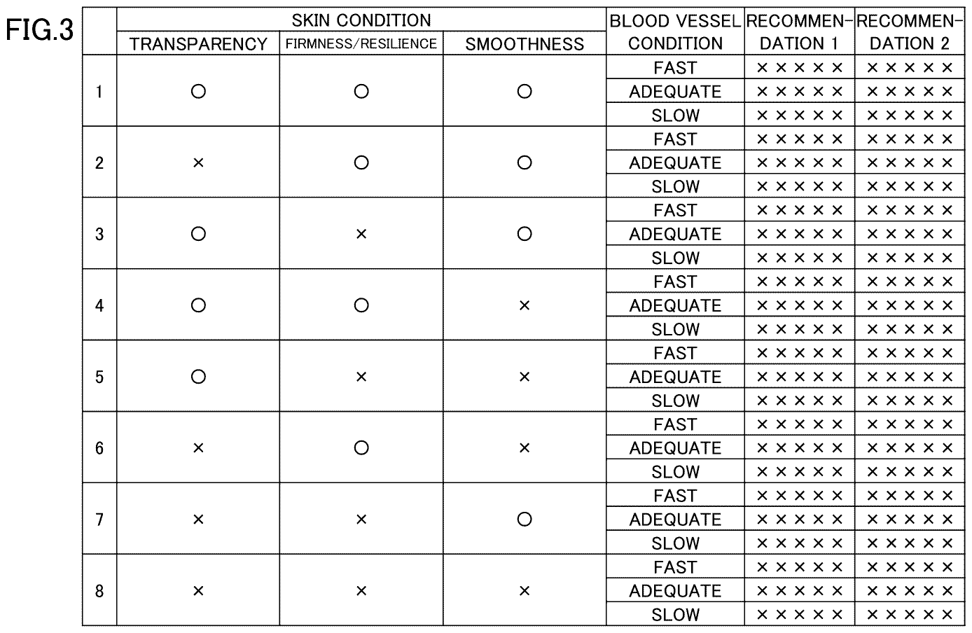

FIG. 3 is an example of information stored in the recommendation information storage unit 107 according to an embodiment of the present invention. The recommendation information storage unit 107 stores information regarding one or a plurality of recommendations corresponding to the skin condition and the blood vessel condition. Each will be described below.

“Skin condition” is the skin condition, such as transparency (blood flow circulation), firmness/resilience (wrinkle/flabbiness), smoothness (skin unevenness), and the like (e.g., good (○), bad (x)).

“Blood vessel condition” is the condition of blood vessels, such as capillary vessels and the like (e.g., the blood flow velocity is fast, the blood flow velocity is adequate, the blood flow velocity is slow, and the like).

“Recommendation” is at least one of a product (e.g., a cosmetic product) or a service (e.g., a beauty service) suitable for the user 20, of whose the skin condition acquired by the skin condition acquisition unit 101 is the condition stored in “Skin condition” and of whose the blood vessel condition acquired by the blood vessel condition acquisition unit 102 is the condition stored in “Blood vessel condition”. For example, a plurality of recommendations may be stored in an order of suitability to the user 20.

The recommendation information storage unit 107 may store a

recommendation based only on the skin condition and a recommendation based on both the skin condition and the blood vessel condition.

### Display Example

An example of a screen image displayed on the skin diagnostic apparatus 10 or the terminal 11 will be described below.

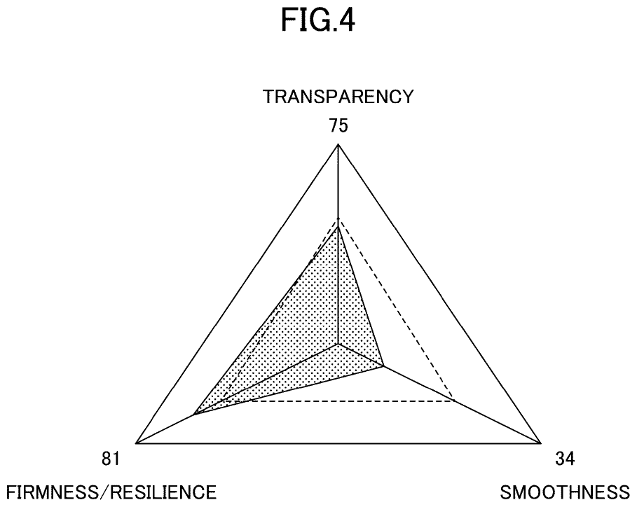

FIG. 4 is a display example of calculated results of the skin indices according to an embodiment of the present invention. For example, the skin index presentation unit 104 can display the skin indices (for example, transparency, firmness/resilience, and smoothness) of the user 20 calculated by the skin index calculation unit 103 based on the skin condition and the blood vessel condition of the user 20 as shown in FIG. 4. In the example shown in FIG. 4, the values of the indices (i.e., transparency: 75, firmness/resilience: 81, smoothness: 34) and a triangular graph representing the values of the indices are displayed.

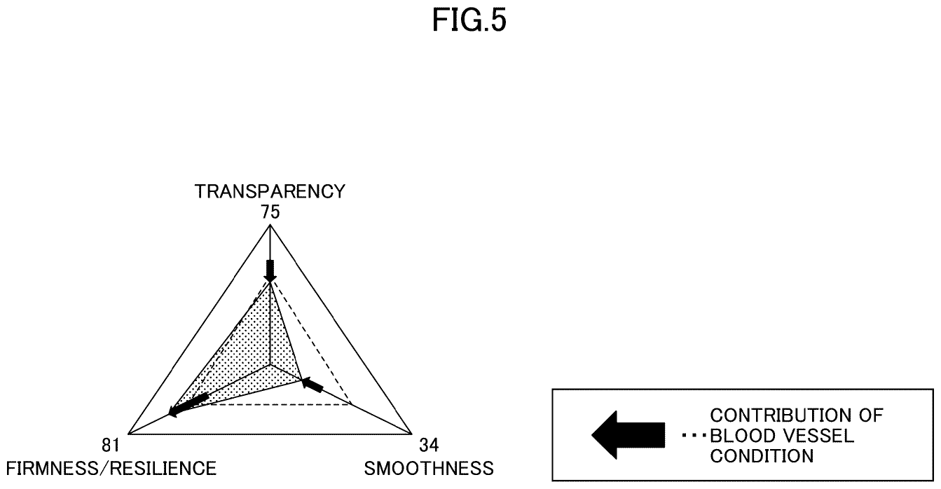

FIG. 5 is a display example of calculated results of the skin indices according to an embodiment of the present invention. In FIG. 5, the contribution of the blood vessel condition to the skin indices of the user 20 is displayed (Example 1).

For example, the skin index presentation unit 104 can display the skin indices (for example, transparency, firmness/resilience, and smoothness) of the user 20 calculated by the skin index calculation unit 103 based on the skin condition and the blood vessel condition of the user 20 and the contribution of the blood vessel condition to each index as shown in FIG. 5. In the example of FIG. 5, the values of the indices (i.e., transparency: 75, firmness/resilience: 81, smoothness: 34) and a triangular graph representing the values of the indices are displayed. Further, in FIG. 5, the degree of the contribution of the blood vessel condition to each index is indicated by an arrow [The example of FIG. 5 shows that the transparency value is low due to the blood vessel condition (i.e., the index would be greater than 75 but for the impact of the blood vessel condition), that the firmness/resilience value is high owing to the blood vessel condition (i.e., the index would be less than 81 but for the contribution of the blood vessel condition), and that the smoothness value is low due to the blood vessel condition (i.e., the index would be greater than 34 but for the impact of the blood vessel condition.)].

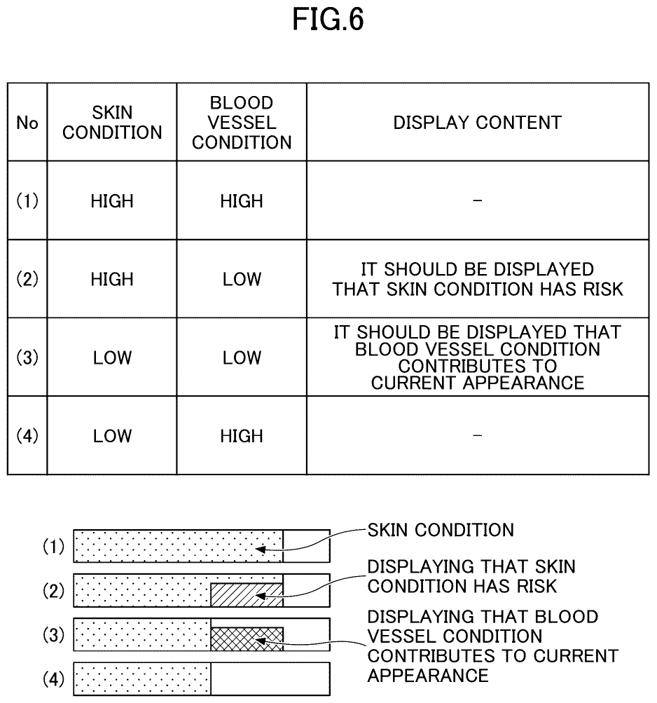

FIG. 6 is a display example of calculated results of the skin indices according to an embodiment of the present invention. In FIG. 6, the contribution of the blood vessel condition to the skin indices of the user 20 is displayed (Example 2).

For example, the skin index presentation unit 104 can display the skin indices (for example, the value of any index of transparency, firmness/resilience, or smoothness) of the user 20 calculated by the skin index calculation unit 103 based on the skin condition and the blood vessel condition of the user 20, and the contribution of the blood vessel condition to each index as shown in FIG. 6.

In FIG. 6, it is assumed that the skin condition score is high and the blood vessel condition score is high in (1), that the skin condition score is high and the blood vessel condition score is low in (2), that the skin condition score is low and the blood vessel condition score is low in (3), and that the skin condition score is low and the blood vessel condition score is high in (4).

In FIG. 6, the contribution of the blood vessel condition to the skin index is displayed as a bar graph. The bar graphs of FIG. 6 indicate that the skin condition score is high (i.e., the current appearance of the skin is good) in (1), that the skin condition score is high (i.e., the current appearance of the skin is good) but that the skin condition has a risk when the blood vessel condition is taken into consideration, together with the degree of the risk in (2), that the skin condition score is low (i.e., the current appearance of the skin is bad) and the blood vessel condition contributes to the current appearance of the skin, together with the degree of the contribution in (3), and that the skin condition score is low (i.e., the current appearance of the skin is bad) in (4). In this way, it is possible to present that the skin condition has a risk in consideration of the blood vessel condition although the current appearance of the skin is good as in (2), and to present the contribution of the blood vessel condition to the current bad appearance of the skin as in (3).

As illustrated in FIGS. 5 and 6, the skin index presentation unit 104 can present the contribution of the blood vessel condition to the skin index. As illustrated in FIG. 6, the skin index presentation unit 104 can present the contribution of the blood vessel condition to the skin index in a display mode corresponding to the skin condition acquired by the skin condition acquisition unit 101 and the blood vessel condition acquired by the blood vessel condition acquisition unit 102.

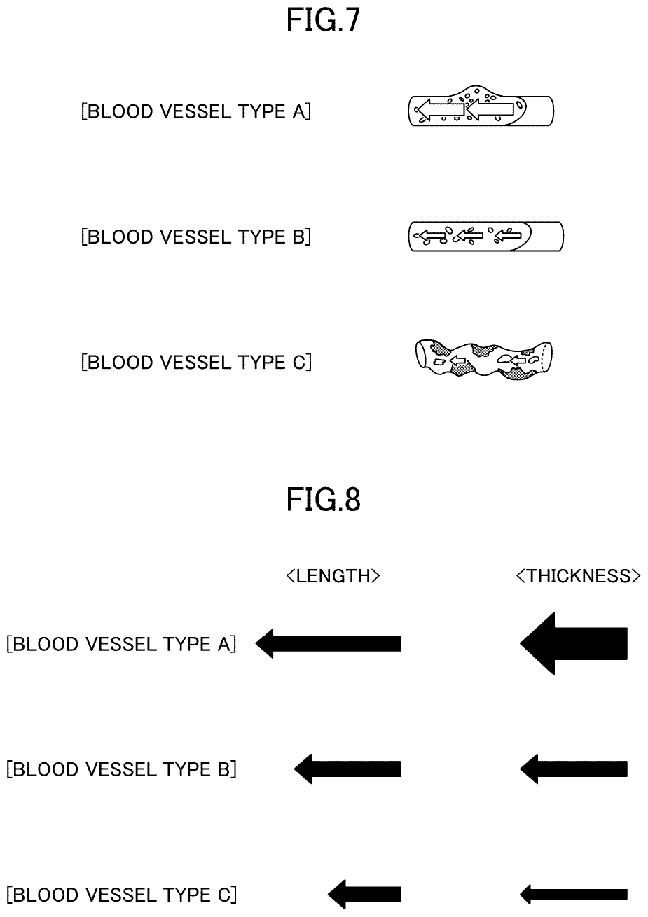

A display example of the blood vessel condition will be described below with reference to FIGS. 7 and 8. For example, it is assumed that there are the following blood vessel types depending on a score that is based on the blood flow velocity of a capillary vessel or the like;
Blood vessel type A when the score based on blood flow velocity is 100 to 70;
Blood vessel type B when the score based on blood flow velocity is 70 to 40; and
Blood vessel type C when the score based on blood flow velocity is 40 to 0.

Too high a score and too low a score representing the blood vessel condition are both unfavorable. Thus, by displaying visual aids as in FIGS. 7 and 8, it is possible to present the blood vessel condition to the user 20 in a way that is easy for the user 20 to understand.

FIG. 7 is a display example of the blood vessel condition according to an embodiment of the present invention. For example, the skin index presentation unit 104 can display a plurality of blood vessel conditions (blood vessel types) by varying a figure simulating a blood vessel.

For example, using the three figures as shown in FIG. 7, the skin index presentation unit 104 can display that the user 20 is a type who has a high blood flow velocity in the blood vessels such as the capillary vessels and the like (blood vessel type A), that the user 20 is a type who has an adequate blood flow velocity in the blood vessels such as the capillary vessels and the like (blood vessel type B), or that the user 20 is a type who has a low blood flow velocity in the blood vessels such as capillary vessels and the like (blood vessel type C).

FIG. 8 is a display example of the blood vessel condition according to an embodiment of the present invention. For example, the skin index presentation unit 104 can display a plurality of blood vessel conditions (blood vessel types) by varying the length of arrows or the thickness of arrows.

For example, the skin index presentation unit 104 can display a long arrow when the user 20 is a type who has a high blood flow velocity in the blood vessels, such as the capillary vessels and the like (blood vessel type A), an arrow having an intermediate length when the user 20 is a type who has an adequate blood flow velocity in the blood vessels such as the capillary vessels and the like (blood vessel type B), and a short arrow when the user 20 is a type who has a low blood flow velocity in the blood vessels such as the capillary vessels and the like (blood vessel type C).

For example, the skin index presentation unit 104 can display a thick arrow when the user 20 is a type who has a high blood flow velocity in the blood vessels such as the capillary vessels and the like (blood vessel type A), an arrow having an intermediate thickness when the user 20 is a type who has an adequate blood flow velocity in the blood vessels such as the capillary vessels and the like (blood vessel type B), and a thin arrow when the user 20 is a type who has a low blood flow velocity in the blood vessels such as the capillary vessels and the like (blood vessel type C).

### <Method>

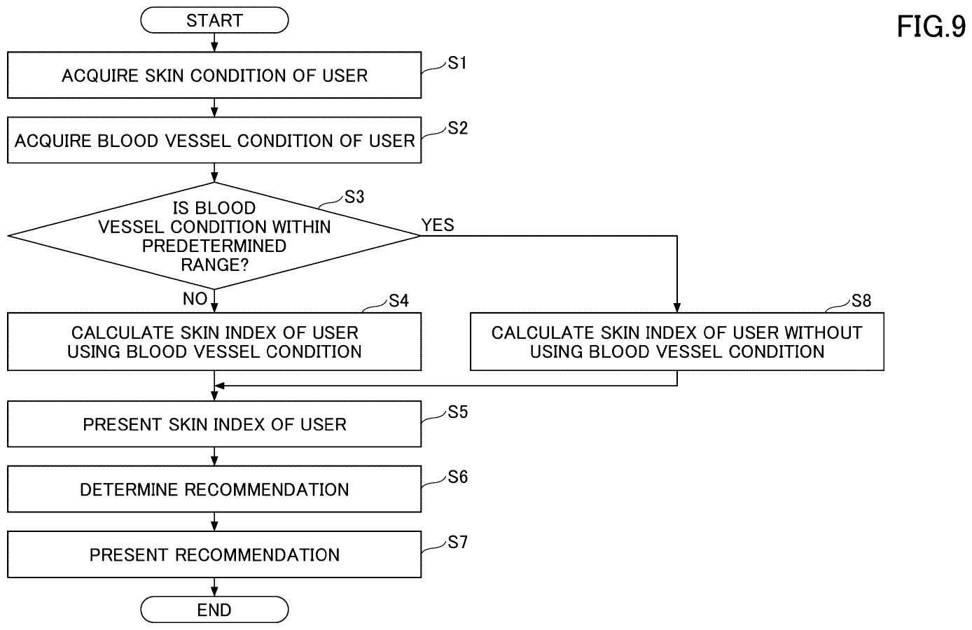

FIG. 9 is a flowchart of a skin diagnosis process according to an embodiment of the present invention.

In step 1 (S1), the skin condition acquisition unit 101 acquires the skin condition of the user 20.

In step 2 (S2), the blood vessel condition acquisition unit 102 acquires the blood vessel condition of the user 20. The order of S1 and S2 may be reversed, or they may be performed simultaneously.

In step 3 (S3), the skin index calculation unit 103 determines whether or not the blood vessel condition acquired in S2 is within the predetermined range. When the blood vessel condition is not within the predetermined range, the flow proceeds to step 4. When the blood vessel condition is within the predetermined range, the flow proceeds to step 8.

In step 4 (S4), the skin index calculation unit 103 calculates the skin index of the user 20 based on the skin condition acquired in S1 and the blood vessel condition acquired in S2.

In step 5 (S5), the skin index presentation unit 104 presents the skin index calculated in S4 or S8 (for example, by displaying on the skin diagnostic apparatus 10 or the terminal 11).

In step 6 (S6), the recommendation determination unit 105 determines a recommendation to be recommended to the user 20 based on both the skin condition acquired in S1 and the blood vessel condition acquired in S2. Alternatively, the recommendation determination unit 105 determines a recommendation to be recommended to the user 20 based only on the skin condition acquired in S1 and a recommendation to be recommended to the user 20 based on both the skin condition acquired in S1 and the blood vessel condition acquired in S2.

In step 7 (S7), the recommendation presentation unit 106 presents the recommendation determined in step S6 to the user 20 (for example, by displaying on the skin diagnostic apparatus 10 or the terminal 11).

S6 and S7 may be performed simultaneously with S4 and S5, respectively, may be reversed with S4 and S5 in the order, respectively, or may be omitted.

In step 8 (S8), the skin index calculation unit 103 calculates the skin index of the user 20 based on the skin condition acquired in step S1.

Then, the flow proceeds to step 5.

### <Effect>

Thus, in one embodiment of the present invention, it is possible to perform diagnoses based not only on the surface condition of the skin of the user 20 but comprehensively on the internal condition of the skin of the user 20. Therefore, it is possible to recommend products and services considering not only the current appearance of the skin of the user 20 but also his/her future skin condition.

### <Hardware Configuration>

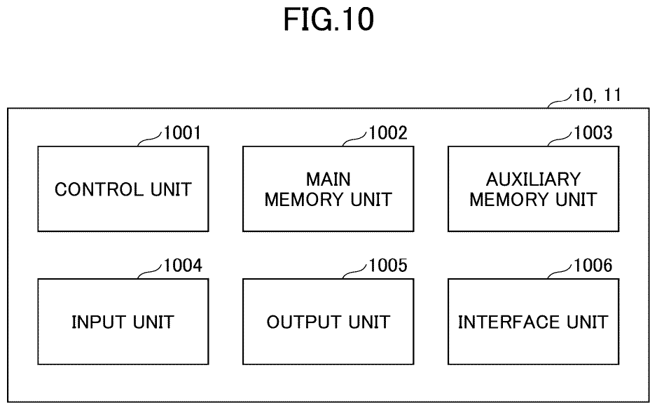

FIG. 10 is a hardware configuration diagram of the skin diagnostic apparatus 10 and the terminal 11 according to an embodiment of the present invention. The skin diagnostic apparatus 10 and the terminal 11 can include a control unit 1001, a main memory unit 1002, an auxiliary memory unit 1003, an input unit 1004, an output unit 1005, and an interface unit 1006. Each will be described below.

The control unit 1001 is a processor (e.g., a Central Processing Unit (CPU), a Graphics Processing Unit (GPU), and the like) that executes various programs installed in the auxiliary memory unit 1003.

The main memory unit 1002 includes a nonvolatile memory (Read Only Memory (ROM)) and a volatile memory (Random Access Memory (RAM)). The ROM stores various programs, data, and the like necessary for the control unit 1001 to execute various programs installed in the auxiliary memory unit 1003. The RAM provides a work area in which the various programs installed in the auxiliary memory unit 1003 are deployed when executed by the control unit 1001.

The auxiliary memory unit 1003 is an auxiliary memory device that stores the various programs and information used when the various programs are executed.

The input unit 1004 is an input device via which an operator of the skin diagnostic apparatus 10 and the terminal 11 inputs various instructions into the skin diagnostic apparatus 10 and the terminal 11.

The output unit 1005 is an output device that outputs an internal state and the like of the skin diagnostic apparatus 10 and the terminal 11.

The interface unit 1006 is a communication device that connects to a network and communicates with other devices.

Although Examples of the present invention have been described in detail above, the present invention is not limited to the specific embodiments described above, and various modifications and changes are applicable within the scope of the spirit of the present invention described in the claims.

This international application claims priority based on Japanese Patent Application No. 2022-203479, filed Dec. 20, 2022, and the entire contents of Japanese Patent Application No. 2022-203479 are incorporated into this international application.

### REFERENCE SIGNS LIST

1 skin diagnostic system
10 skin diagnostic apparatus
11 terminal
20 user
101 skin condition acquisition unit
102 blood vessel condition acquisition unit
103 skin index calculation unit
104 skin index presentation unit
105 recommendation determination unit
106 recommendation presentation unit
107 recommendation information storage unit
1001 control unit
1002 main memory unit
1003 auxiliary memory unit
1004 input unit
1005 output unit
1006 interface unit

1. A skin diagnostic system comprising:
a processor; and
a memory,
wherein the processor is configured to:
acquire a skin condition of a user;
acquire a blood vessel condition of the user; and
calculate a skin index of the user based on the skin condition acquired and the blood vessel condition acquired.

2. The skin diagnostic system according to claim 1,
wherein the processor calculates the skin index of the user based on the skin condition acquired and the blood vessel condition acquired, when the blood vessel condition is outside a predetermined range.

3. The skin diagnostic system according to claim 2, wherein the processor sets the predetermined range.

4. The skin diagnostic system according to claim 1,
wherein the processor is further configured to present the skin index to the user,
wherein the processor presents contribution of the blood vessel condition to the skin index.

5. The skin diagnostic system according to claim 1,
wherein the processor is further configured to present the skin index to the user,
wherein the processor presents contribution of the blood vessel condition to the skin index in a display mode corresponding to the skin condition acquired and the blood vessel condition acquired.

6. The skin diagnostic system according to claim 1,
wherein the processor is further configured to determine a recommendation to be recommended to the user based on both the skin condition acquired and the blood vessel condition acquired.

7. The skin diagnostic system according to claim 1,
wherein a recommendation to be recommended to the user based only on the skin condition acquired, and a recommendation to be recommended to the user based on both the skin condition acquired and the blood vessel condition acquired are determined.

8. The skin diagnostic system according to claim 6,
wherein the processor is further configured to present the user with the recommendation determined.

9. The skin diagnostic system according to claim 7,
wherein whether to determine the recommendation based only on the skin condition acquired or to determine the recommendation based on both the skin condition acquired and the blood vessel condition acquired is selected by the user.

10. The skin diagnostic system according to claim 1,
wherein the skin index includes indices of a plurality of skin factors, and
the processor calculates the indices of the skin factors in accordance with degrees of correlations between the skin factors and the blood vessel condition, respectively.

11. The skin diagnostic system according to claim 1,
wherein the blood vessel condition is at least one of blood flow in capillary vessels, number of capillary vessels, density of capillary vessels, number of capillary vessel branches, distance from a base layer of an epidermis to capillary vessels, or thickness of the epidermis.

12. (canceled)

13. A non-transitory computer-readable recording medium storing a program causing a skin diagnostic apparatus to:
acquire a skin condition of a user;
acquire a blood vessel condition of the user; and
calculate a skin index of the user based on the skin condition acquired and the blood vessel condition acquired.

14. A method executed by a skin diagnostic apparatus, the method comprising:
acquiring a skin condition of a user:
acquiring a blood vessel condition of the user; and
calculating a skin index of the user based on the skin condition acquired and the blood vessel condition acquired.

15. The skin diagnostic system according to claim 1,
wherein the skin diagnostic system is an information processing system comprising a terminal and a server.

16. The skin diagnostic system according to claim 1,
wherein the skin diagnostic system comprises a skin diagnostic apparatus, and
wherein the skin diagnostic apparatus includes the processor and the memory.

17. The skin diagnostic system according to claim 1,
wherein the skin condition of the user is acquired by image analysis, and
wherein the blood vessel condition of the user is acquired by a model.
